(19) United States
(12) Patent Application Publication
(10) Pub. No.: US 2025/0327061 A1
(43) Pub. Date: Oct. 23, 2025

(54) SYSTEMS AND METHODS FOR ISOLATING A TARGET FROM A FLUID SAMPLE

(71) Applicants: Salus Discovery, LLC, Madison, WI (US); Flambeau Diagnostics LLC., Madison, WI (US)

(72) Inventors: Jay Warrick, Madison, WI (US); Patrick McMinn, Madison, WI (US); Ryan Shogren, Madison, WI (US); David Beebe, Madison, WI (US)

(21) Appl. No.: 18/851,200

(22) PCT Filed: Mar. 29, 2023
(86) PCT No.: PCT/US2023/016674

§ 371 Date: Sep. 26, 2024

Related U.S. Application Data
(60) Provisional application No. 63/324,924, filed on Mar. 29, 2022.

(51) Int. Cl. C12N 15/10; B01L 3/02; B03C 1/01; B03C 1/28; G01N 33/543

(52) U.S. Cl. CPC C12N 15/1013; B01L 3/021; B03C 1/01; B03C 1/288; G01N 33/54333; B01L 2200/0631; B01L 2400/043; B03C 2201/18; B03C 2201/26

(57) ABSTRACT

Provided herein are systems and methods for isolating, extracting or separating at least one target or component from a sample or a fluid comprising a sample. In some aspects, provided herein are systems and methods for isolating at least one target or component from a sample or a fluid comprising a sample by diffusion of particles from the sample or fluid comprising a sample to a destination fluid. In some aspects, provided herein are systems and methods for isolating at least one target from a fluid sample by drawing a particle (e.g. a target, a contaminant) bound to a magnetic particle (e.g.) a paramagnetic particle) into a destination fluid through application of a magnetic force. In some aspects, provided herein are systems and methods where a target or sample component or a complex comprising a magnetic particle and a target or sample component is transferred to another or different device or reagent mixture or reagent for further processing and/or analysis, identification or quantification.

### STATEMENT REGARDING RELATED APPLICATIONS

This application claims priority to U.S. Provisional Patent Application No. 63/324,924, filed Mar. 29, 2022, the entire contents of which are incorporated herein by reference for all purposes.

### INCORPORATION BY REFERENCE

All U.S. patents, U.S. patent applications, publications, foreign patents, foreign and PCT published applications, articles and other documents, references and publications noted herein, and all those listed as References Cited in any patent or patents that issue herefrom, are hereby incorporated by reference in their entirety. The information incorporated is as much a part of this application as if all the text and other content is repeated in the application and will be treated as part of the text and content of this application as filed.

### TECHNICAL FIELD

The invention generally concerns the extraction, separation, and isolation of materials. Provided herein are systems and methods for extracting, separating or isolating a target. In some aspects, provided herein are systems and methods for isolating a target from a sample or a fluid comprising a sample by a natural transport process (e.g. diffusion, convection, etc.) that moves a target from a sample or a fluid comprising a sample to a destination fluid. In some aspects, provided herein are systems and methods for extracting or isolating a target from a sample, e.g., a fluid sample, by use of an active force (e.g. centrifugation, magnetism, etc.) that can comprise drawing one or more targets and/or one or more contaminants bound to a magnetic particle into a destination fluid through application of a magnetic force, for example.

### BACKGROUND

The ability to extract or separate or isolate a target (e.g., nucleic acid, protein, whole cell) from a complex background is a critical prerequisite for many common analytical processes in diagnostics, biological research, biomarker discovery, forensics, and more. However, conventional target isolation processes are time-consuming, expensive, and laborious, often becoming the bottleneck within the analytical process.

### BRIEF SUMMARY

The inventions described and claimed herein have many attributes and embodiments including, but not limited to, those set forth or described or referenced in this Brief Summary. It is not intended to be all-inclusive, and the inventions described and claimed herein are not limited to or by the features or embodiments identified in this introduction, which is included for purposes of illustration only and not restriction.

Some prior methodologies for the extraction or separation or isolation of a target can cause damage to the sample or result in undesired loss or inconsistent yield of sample. Moreover, some methodologies are accompanied by significant levels of unwanted carryover during isolation of the target, resulting in contamination which can jeopardize downstream applications, such as assays for detection of the analyte. Accordingly, improved methods and systems for the extraction, separation and/or isolation of a target from a sample are needed and are provided herein.

Other prior methodologies involving the use of beads (e.g. paramagnetic particles) for the extraction, separation and/or isolation of a target from a sample result in incomplete bead movement and/or capture for analysis. Improved methods and systems for the extraction, separation and/or isolation of a target from a sample in which no beads or bead packets are left behind in carrying out one or more of these methods are provided herein, including extraction/separation/isolation methods that allow all beads and bead packets to be moved, accounted for, and counted.

The invention methods and devices and systems of the invention can be used to extract, separate or isolate a target from a sample for transfer to another device or reagent mixture or reagent for further processing and/or analysis, identification, quantification or use.

In some aspects, provided herein are methods and systems for extracting and/or isolating at least one target from a fluid sample or from a fluid comprising a sample. In some aspects, methods and systems are provided for extracting and/or isolating at least one target from a fluid sample or from a fluid comprising a sample for processing. In some aspects, methods and systems are provided for extracting and/or isolating at least one target from a fluid sample or from a fluid comprising a sample for transfer of the target to another device for processing. In some embodiments, the target is an analyte and the processing or the device for processing is an assay. In some embodiments, the assay is used to qualitatively assess or quantitatively measure the presence, amount, or functional activity of the target. In some aspects, methods and systems are provided for processing and presenting at least one target from a fluid sample or from a fluid comprising a sample for assay. In some embodiments, a method or system of the invention includes an assay for detecting, identifying and/or determining the presence or amount of a target or analyte.

In some embodiments, the method comprises placing a container housing the sample into a destination fluid. In some embodiments, the container comprises an opening that permits movement of particles from the sample to the destination fluid. In some embodiments, the sample is sufficiently miscible with the destination fluid. In some embodiments, a particle that moves from the sample to the destination fluid is a target (e.g. a cell, an analyte, etc.). In some embodiments the opening permits movement of at least one target from the sample to the destination fluid. In some embodiments, a particle that moves from the sample to the destination fluid is a contaminant. In some embodiments, the opening permits movement of at least one contaminant from the sample to the destination fluid. In some embodiments, movement of particles (e.g. movement of the at least one target, movement of the at least one contaminant, or both) from the sample to the destination fluid occurs naturally, i.e., without application of a force (e.g. through diffusion, osmosis, gravity, random walk, etc.), or by other means (e.g. acceleration, filtration, centrifugation, dynamic dialysis, electrophoresis, etc.). In some embodiments, a combination of one or more methods of movement by natural and/or applied forces may be utilized, serially or simultaneously.

In some embodiments, the method further comprises removing fluid from the destination fluid into the container after the container is placed into the destination fluid (e.g., by aspiration, capillary action, etc.). For example, fluid may be aspirated or otherwise moved into the container before, during, and/or after movement of particles from the container to the destination fluid.

In some embodiments, the at least one target, if present in the sample, is bound to at least one paramagnetic particle (PMP) to form one or more target-PMP complexes. In some embodiments, the opening permits movement of the one or more target-PMP complexes from the sample to the destination fluid. In some embodiments, movement of the one or more target-PMP complexes from the sample to the destination fluid occurs through application of a magnetic force to draw the one or more target-PMP complexes from the sample into the destination fluid.

In some embodiments, the at least one contaminant, if present in the sample, is bound to one or more paramagnetic particles (PMPs), to form one or more contaminant-PMP complexes. in some embodiments, the opening permits movement of the one or more contaminant-PMP complexes from the sample into the destination fluid. In some embodiments, movement of the one or more contaminant-PMP complexes from the sample to the destination fluid occurs through application of a magnetic force to draw the one or more contaminant-PMP complexes from the sample into the destination fluid.

In some embodiments, the method comprises applying a magnetic force to draw the one or more target-PMP complexes or the one or more contaminant-PMP complexes from the sample into the destination fluid, and the method further comprises aspirating fluid into the container before, during, and/or after application of the magnetic force.

In some embodiments, the method further comprises generating a pocket of air, oil, or gas proximal to the opening of the container prior to inserting the container into the destination fluid.

In some embodiments, the at least one target is a cell. In some embodiments, the at least one target is an analyte. In some embodiments, the sample is a biological fluid. In some embodiments, the container is a pipette tip, a straw, or a capillary tube.

In some embodiments, the destination fluid is a wash buffer, an elution buffer, or a reaction mixture comprising reagents for detecting the target. The destination fluid can be anything sufficiently miscible with a sample or with the fluid in the container, and also includes, for example, extraction buffer, water, saline, precipitating buffer, etc.

In some embodiments, the method further comprises placing the container into a cleansing fluid to reduce potential contaminants on an exterior surface of the container prior to placing the container into the destination fluid.

In some aspects, provided herein are systems for isolating at least one target from a fluid sample. In some embodiments, the system optionally comprises a fluid sample comprising the at least one target. In some embodiments, the system comprises a container housing the fluid sample, wherein the container comprises an opening. In some embodiments, the system comprises a collection device comprising a destination fluid. In some embodiments, the sample or a fluid comprising a sample is sufficiently miscible with the destination fluid.

In some embodiments, the fluid sample comprises the at least one target bound to one or more paramagnetic particles, thus forming one or more target-PMP complexes. In some embodiments, the fluid sample comprises at least one contaminant bound to one or more paramagnetic particles, thus forming one or more contaminant-PMP complexes. In some embodiments, the system further comprises a magnet.

In some embodiments, the system further comprises a cleansing fluid. In some embodiments, the cleansing fluid is housed within the collection device. In some embodiments, the cleansing fluid is housed within the collection device, and the cleansing fluid resides on top of the destination fluid such that the container housing the sample passes through the cleansing fluid prior to contacting the destination fluid. In some embodiments, the cleansing fluid is an oil or other layer that resides on top of the destination fluid. In some embodiments, the cleansing fluid and the destination fluid are immiscible. In some embodiments, the cleansing fluid is not housed within the collection device.

In some embodiments, the at least one target is a cell. In some embodiments, the at least one target is an analyte. In some embodiments, the sample is a biological fluid. In some embodiments, the container is a pipette tip, a straw, or a capillary tube. In some embodiments, the destination fluid is a wash buffer, an elution buffer, or a reaction mixture comprising reagents for detecting the target.

In a method of isolating at least one target from a fluid sample, the method comprises placing a container housing the sample into a destination fluid, wherein the container comprises an opening that permits movement of particles from the sample to the destination fluid, and the sample is sufficiently miscible with the destination fluid. In some embodiments of the method, the opening permits movement of at least one target from the sample to the destination fluid. In some embodiments of the method, the opening permits movement of at least one contaminant from the sample to the destination fluid. In some embodiments of these methods, the movement of the particles from the sample to the destination fluid occurs through diffusion, gravity, or acceleration. In some embodiments of these methods, the methods further comprise aspirating fluid into the container after the container is placed into the destination fluid, and the fluid may be aspirated into the container before, during, and/or after movement of the particles from the container to the destination fluid. In some embodiments of these methods, the at least one target, if present in the sample, is bound to at least one paramagnetic particle (PMP) to form one or more target-PMP complexes. In some embodiments, the least one contaminant, if present in the sample, is bound to one or more paramagnetic particles (PMPs), to form one or more contaminant-PMP complexes. In some embodiments, the opening permits movement of the one or more target-PMP complexes and/or contaminant-PMP complexes from the sample to the destination fluid. In some embodiments, the movement of the one or more target-PMP complexes and/or contaminant-PMP complexes from the sample to the destination fluid occurs through application of a magnetic force to draw the one or more target-PMP complexes from the sample into the destination fluid. In some embodiments of these methods, the method further comprises aspirating fluid into the container before, during, and/or after application of the magnetic force. In any of these methods, the method may further comprise generating a pocket of air, oil, or gas proximal to the opening of the container prior to inserting the container into the destination fluid. In some embodiments of these methods, the at least one target is a cell. In some embodiments of these methods, the at least one target is an analyte. In some embodiments of these methods, the sample is a biological fluid, a biological sample or an environmental sample. In some embodiments of these methods, the container is a pipette tip, a straw, or a capillary tube or like device. In some embodiments of these methods, the destination fluid is or comprises a wash buffer, an elution buffer, or a reaction mixture comprising reagents for detecting the target and/or the contaminant. In any of these methods, the method may further comprise placing the container into a cleansing fluid to reduce potential contaminants on an exterior surface of the container prior to placing the container into the destination fluid.

The inventions also include systems, including systems for isolating at least one target from a fluid sample or a sample comprising a fluid where the system comprises a container housing the fluid sample, wherein the container comprises an opening, and a collection device comprising a destination fluid, wherein the destination fluid the is sufficiently miscible with the fluid sample or the sample comprising a fluid. In some embodiments, the system may further comprise a fluid sample comprising the at least one target (or suspected of comprising the at least one target). In some embodiments, the fluid sample comprises the at least one target bound to one or more paramagnetic particles, thus forming one or more target-PMP complexes. In some embodiments, the fluid sample comprises at least one contaminant bound to one or more paramagnetic particles, thus forming one or more contaminant-PMP complexes. In some embodiments, the system further comprises a magnet or other means for creating a magnetic force. In some embodiments, the system further comprises a cleansing fluid (and/or means for holding a a cleansing fluid). In some embodiments, the cleansing fluid is housed within the collection device, wherein the cleansing fluid resides on top of the destination fluid such that the container housing the sample passes through the cleansing fluid prior to contacting the destination fluid. In some embodiments, the cleansing fluid and the destination fluid are immiscible. In some embodiments of the systems, the at least one target is a cell or an analyte. In some embodiments of the systems, the sample is a biological fluid, a biological sample, or an environmental sample. In some embodiments of the systems, the container is a pipette tip, a straw, or a capillary tube or like device. In some embodiments of these systems, the destination fluid is or comprises a wash buffer, an elution buffer, or a reaction mixture comprising reagents for detecting the target and/or the contaminant.

### BRIEF DESCRIPTION OF THE DRAWINGS

FIG. 1 shows a schematic of one embodiment of the methods described herein. The schematic demonstrates isolation of a target bound to paramagnetic particles to produce one or more target-PMP complexes. The target-PMP complexes are housed within a container comprising an opening (1). The container is placed into a destination fluid, which in this example is a wash solution (2). A magnetic force is applied, thereby drawing target-PMP complexes out of the container (3). In addition, the sample will diffuse into the destination fluid, due to the miscibility of the two fluids. The distance of diffusion around the container can be calculated (4). The volume of diffusion can be calculated based upon the distance of diffusion, and this volume can be moved (e.g. aspirated) back into the container (5), thereby significantly reducing contamination within the destination fluid.

FIG. 2 shows a schematic of another embodiment of the methods described herein. In some embodiments, an air pocket (e.g. “bubble”) may be generated within the container housing the fluid sample. The air bubble is generated prior to inserting the container into the destination fluid. After inserting the container into the destination fluid (in this instance, a wash fluid), the wash fluid is moved (e.g. by aspiration) into the container. Aspiration forces the air pocket (e.g. bubble) to rise within the container with the air bubble initially positioned at the interface of the two fluids, thereby permitting contact between the fluid sample and the destination fluid yet restricting the area of the initial interface between the two fluids. Following contact of the two fluids, transfer of the target from the sample to the destination fluid can occur around the bubble, through the sufficiently miscible interface between the two fluids. This can be used, for example, to restrict diffusion-based transport of contaminants or other undesired materials.

FIG. 3 shows results from an experiment to quantify contamination resulting from carry-over of contaminants from the outside surface of the pipette tip into the destination fluid (e.g. “carry-over contamination”). A solution of tartrazine (a yellow dye) and paramagnetic particles (PMPs) was aspirated into pipette tips and then plunged into wells containing double-distilled water (ddH2O) with a magnet below it. The beads were allowed to move out of the pipette tip and into the well. Once all of the beads were translocated into the well, the tips were removed. The beads in the well were then removed (via magnet) and the absorbance of tartrazine in the destination fluid was taken (i.e. measured by spectrophotometry). % carryover of tartrazine was then calculated. FIG. 4 and FIG. 5 show results from two experiments comparing carryover contamination of various techniques. “Tube-It” refers to mimicking washing of beads in a tube where beads are held on the side of the tube with a magnetic force while fluid is added and removed. Similarly, “Tube-It” refers to holding beads in the bottom of a 96-well plate with a magnet while fluid is added and removed. “Fuse-It” refers to the minimal approach where a device suitable for containing and manipulating a sample (e.g. a pipette tip) is brought into the destination well and removed. “Pre-Wash-Aspiration” and “Post-Wash-Aspiration” refer to (1) aspiration of wash solution into a device suitable for containing and manipulating a sample (e.g. a pipette tip) prior to transfer of the target/beads from the sample to the destination fluid (pre-aspiration) or (2) aspiration of wash solution into a device suitable for containing and manipulating a sample (e.g. a pipette tip) following transfer of the target/beads from the sample to the destination fluid (post-aspiration). An example of an enhanced Fuse-It procedure includes “Bubble-It,” which refers to (3) the pre-air-aspiration into the pipette tip prior to inserting the tip into the destination fluid, as described in FIG. 2. In some embodiments, steps included in Bubble-It and Fuse-It may be used together, in combination (see FIG. 2, last panel). The phrase “Tip-Dip” refers to dipping a device suitable for containing and manipulating a sample (e.g. a pipette tip) briefly into a sacrificial well of, for example, wash fluid prior to entering the final destination fluid as a means to reduce carryover of surface contamination. In particular, FIG. 4 compares performance of Tube-It with variations of Fuse-It. Data demonstrate that Fuse-It with both pre- and post-aspiration can provide the same wash benefit as a single Tube-It wash, regardless of whether the beads are pre-concentrated to the end of, for example, a pipette tip prior to entry into the wash fluid. Data also demonstrate the sizable benefit provided when Fuse-It is enhanced by Bubble-It. With the Bubble-It procedure included in Fuse-It, washing is >4 times better than Tube-It alone. After two washes (i.e. carrying out Fuse-It with Bubble-It two times with one sample), Fuse-It with Bubble-It is ˜16 times better (i.e., 4×4) than 2 Tube-It washes. FIG. 5 illustrates, in a separate experiment, the benefit of removing surface carryover when performing Fuse-It and again confirms the relative benefit of Fuse-It compared to Tube-It (FIG. 5, Bar 1 vs. Bar 2). ˜30% of the contamination from Fuse-It with Bubble-It is from surface carryover on the outside of the tip as shown by results with and without Tip-Dip (FIG. 5, Bar 2 vs. Bar 3). In both FIG. 4 and FIG. 5, “Carryover (%)” refers to the percent carryover of tartrazine as measured by spectrophotometry.

FIG. 6 compares the timing breakdown of running the same protocol for extracting nucleic acids with the KingFisher magnet in a sheath technology and the methods of the invention, in this case, Fuse-It with Pipetmax, showing that methods of the invention are twice as fast as the KingFisher technology (i.e., the “Technique” portion of overall time). In the Figure legends, Tip Management refers to loading and ejecting tips. Fluid Handling refers to well mixing. Technique refers to bead collection and transfer.

FIG. 7 shows several variables that improve bead transfer from a pipette tip with Fuse-It and their impact on carryover.

FIG. 8 shows a purity comparison between KingFisher magnet in a sheath technology and the TSP platform using a Macherey Nagel Nucleomag Tissue kit. TSP refers to tip-based sample preparation, which encompasses methods of the invention including Tip-It, Fuse-It and Bubble-It. The 24 uL TSP method is roughly equivalent to the KingFisher in carryover. The 10 uL TSP platform using methods of the invention results in 10× less carryover.

### DEFINITIONS

To facilitate an understanding of the present disclosure, a number of terms and phrases are defined below:

As used herein, the term “container” means any device, receptacle, or vessel capable of holding material comprising a sample and includes any device, receptacle, or vessel in which a sample may be housed, stored or held and used in a method of the invention. In some embodiments, the container is a device capable of being used to perform a pre-aspiration step, a post-aspiration step, or a Bubble-It procedure, as described herein. In some embodiments, the container is a pipette or pipette-type device. In some embodiments, the container is portable. In some embodiments, the container is a disposable or single-use container. In some embodiments, the container is reusable. In some embodiments, the container is a part of an automated or semi-automated system comprising elements to accomplish a method of the invention and operate the system. Vessels and containers include, for example, any vessel, container, receptacle, holder, carrier, cartridge, or storage device capable of holding material comprising a sample. Containers provide for movement of fluids during use of the systems and methods of the invention. In some embodiments, a container comprises a top opening to permit addition of a sample to the container. In some embodiments, the container includes materials comprising a lysis buffer, a wash buffer or an extraction buffer, or other materials, for example. In some embodiments, the container may include a filter, a mesh or any useful porous or other material to provide for pre-filtering of sample contaminants before or during insertion of the container into the destination fluid when carrying out a method or operating a system of the invention. In some embodiments, the container comprises one or more openings to allow removal of target or target-binding particles (e.g., PMPs) from a sample (e.g., using a magnet). In some embodiments, contaminant-binding particles (e.g., PMPs) are removed from a sample (e.g., using a magnet) through the one or more openings and the target or target-binding particles remaining in the container are moved into another vessel or destination (e.g., a multi-well plate), or into or onto a detector (e.g., a reader, a blue-tooth enabled reader or instrument, etc.) that can accept the target or target-binding particles, or onto or into a surface or porous material (e.g., a spot card for drying and transport of sample for later analysis, etc.).

As used herein, the term “carryover” is used to describe the transfer of unwanted material from one location to another (e.g. from one container or mixture to another). Carryover, when used in reference to removal of contaminants or unwanted or undesired or undesirable material, refers to the transfer of unwanted or undesired or undesirable material from one location to another (e.g., from one container to another, or one fluid to another, or one fluid to a container, or one container to a fluid, or one region to another region, etc.). Carryover may be used in reference to and action, e.g., “reduce carryover of unwanted material” or to the unwanted material itself (e.g., “the concentration of carryover in the final reaction”).

As used herein, the term “destination container” refers to any device, receptacle, or vessel capable of holding a destination fluid and receiving a container. In some embodiments of the method and/or system, the destination container is initially empty, and later accepts a destination fluid, for example when a container housing a sample is brought into proximity with a surface and a small volume of fluid is dispensed, passively or actively, from the container to generate a destination fluid on the surface.

As used herein, the term “destination fluid” means a fluid that is sufficiently miscible with a fluid in a container (e.g. a fluid comprising a sample). In some embodiments, the destination fluid comprises one or more of a wash buffer, an elution buffer, a precipitating buffer, water, saline and a reaction mixture comprising reagents for detecting a target. In some embodiments, the destination fluid is heated to a temperature higher than the material comprising a sample in the container (e.g. to aid in movement by convection). In some embodiments, the destination fluid is selected or configured to allow for movement of a material comprising a sample down a pressure gradient (e.g. a pressure gradient from a container comprising a sample to and/or through a destination fluid in a destination container).

As used herein, the term “magnetic particle” refers to a particle material (e.g. a micro- or nano-material) that displays magnetic properties when subjected to a magnetic field (e.g., an external or internal magnetic field). In some embodiments, a magnetic particle comprises two components, a magnetic material (e.g. iron, nickel, cobalt, etc.) and a chemical component that has functionality (e.g. an antibody or antibody binding fragment). In some embodiments, a magnetic particle is polymer-functionalized, amine-functionalize, aldehyde-functionalized, surfactant-functionalized, ligand-functionalized, etc. For use in the methods and systems of the invention, magnetic particles will be selected to have a particle size or mean particle size as desired (e.g. depending on the size of target, the size of container openings, the inclusion and use of a pre-filter of a particular size, etc.). Magnetic particles include particles comprising paramagnetic materials (e.g. paramagnetic particles (PMPs), paramagnetic microspheres, etc.). Paramagnetic material include material comprising paramagnetic atoms (e.g. aluminum, iron oxide, etc.). Magnetic particles include particles comprising ferromagnetic materials (e.g. ferromagnetic particles).

A “magnet” for use in a system, device or method of the invention refers to a means for generating magnetic force. As used herein, magnets include permanent magnets, temporary magnets and electromagnets.

As used herein, the term “cleansing fluid” means any fluid useful for removing or isolating contaminating fluid or unwanted material from a surface of a container to prevent it (in whole or in part) from entering or contaminating a destination fluid. For example, a “cleansing fluid” includes a separate wash buffer well for dipping a container, an oil overlay covering a destination fluid to exclude or help remove contaminating fluid from the surface of a container, etc.. A cleansing fluid may be a fluid that mimics a destination fluid, and may be in a destination fluid or maintained separately elsewhere in a system of the invention.

As used herein, the term “oil layer” means a layer in a system of the invention that compromises an oil and is substantially hydrophobic and does not substantially mix with an aqueous layer (e.g. a destination fluid). Suitable oil layers in some systems of the invention include, for example, mineral oil, coconut oil, vegetable oil. Other oils include carbon- and silicone-based polymeric compounds, mineral oils, silicone oils, paraffin waxes, and fluorinated oils, for example. As used herein, the term “oil” refers to any of numerous substances, usually liquid or semi-solid substances, that do not dissolve in water. Oils also include mixtures of oils (e.g. waxes with different melting temperatures; polymeric oils with different chain lengths; mineral oil and silicone oil; etc.). Oils also include oil-oil emulsions.

As used herein, the term “interface” means a surface forming a common boundary or transition zone between adjacent regions, bodies, substances, phases or layers (e.g. the boundary between an oil layer and a destination fluid).

As used herein, the term “porous” means having pores or other small spaces that can allow a target (or non-target, e.g. a contaminant) whether bound or unbound to, or part of, a solid phase or other carrier to pass through, or not pass through, as desired. Reference to “porous material” or structure, or to a “porous mesh” or “porous layer” means a material comprising void spaces, i.e., spaces not occupied by the main framework of atoms that make up the structure of the material. A material through which a target (bound or unbound to a solid phase) to pass through is an example of a porous material. A material through which non-target materials but not a target (bound or unbound to a solid phase) do not pass through is also an example of a porous material. A porous material or structure, a porous mesh or a porous layer does not need to be constructed of, or consists of, a single material, i.e., it does not need to be homogeneous. A porous material or structure, a porous mesh or a porous layer for use in systems, devices, methods and compositions of the invention may comprise different materials, i.e., it may be heterogeneous or inhomogeneous (e.g., in one embodiment, comprising polystyrene and nylon or spatially variable mixtures).

As used herein, the terms “detect”, “detecting”, or “detection” may describe either the general act of discovering or discerning or the specific observation of a detectably labeled composition. The term “detecting” when used in reference to a target refers to detecting either the presence or the absence or the amount of the target in the sample. In some embodiments, detecting may be qualitative, semi-quantitative, or quantitative. In some embodiments, “detecting” a target in a sample refers to determining that the target is present in the sample. In some embodiments, “detecting” a target in a sample refers to determining that the target is not present in the sample or is not present in sufficient quantities to be detected in the sample. In some embodiments, “detecting” a target in a sample refers to determining the amount of target is present in a sample, or that the amount of target is present in a sample is at or above a threshold amount.

The term “fluid” is used herein in the broadest sense and refers to any substance that flows. In some embodiments, a fluid is a liquid. For example, a “fluid sample” may refer to a liquid sample. In other embodiments, a fluid is a gas.

The term “sample” as used herein is used in the broadest sense and is inclusive of many sample types. In some embodiments, the sample is a “biological sample.” In other embodiments, the sample may be an “environmental sample.” In some embodiments, a sample will refer to a portion of material taken or selected from a larger quantity of material. In some embodiments, sample refers to any material containing or suspected of containing a target. In some embodiments, the sample is an entire quantity of material, e.g., blood. In some embodiments, the sample is blood, cerebrospinal fluid, urine, tissue, biopsy tissue, etc. Any type of fluid comprising a sample containing or suspected of containing a target (and/or contaminant) of interest is contemplated for use in the systems and methods of the invention.

In some embodiments, a fluid sample is a biological sample. As used herein, the term “biological sample” is used in the broadest sense and is inclusive of many sample types that may be obtained from a subject. Biological samples may be obtained from animals (including humans) and encompass fluids (e.g. urine, blood, blood products, sputum, saliva, cerebrospinal fluid, etc.), solids, tissues, and gases. Biological samples include saliva, blood products, such as plasma, serum and the like. In some embodiments, the biological sample is a nasopharyngeal sample, an oropharyngeal sample, oral swab or sponge sample, a nasal swab sample, a mid-turbinate sample, or a saliva sample. In some embodiments, the biological sample is a blood sample, a serum sample, or a plasma sample. In some embodiments, the biological sample is a saliva sample. The term “saliva sample” as used herein refers to a sample of saliva from a subject, or collected from a subject. In some embodiments, the biological sample is a nasopharyngeal (NP) sample. A “nasopharyngeal sample” refers to a specimen collected using a swab inserted into the nasopharyngeal cavity of a subject.

The biological sample may be subjected to various pretreatment steps prior to performing a method as described herein. For example, the biological sample may be frozen, heated, mixed with various denaturants (e.g. guanidium thiocyanate), mixed with viscosity reducing reagents (e.g. DTT), mixed with inhibitors of target degradation (e.g. protease inhibitors, RNAse inhibitors, etc.), mixed with various buffers, or subjected to other suitable pre-treatment steps. Any of the substances added to the biological sample (e.g. denaturants, viscosity reducing reagents, inhibitors of target degradation, buffers, etc.) may be added to the biological sample or may be present in a storage buffer present in a container into which the sample is collected (e.g. present within a storage buffer in a sample collection tube). In some embodiments, samples contain or are suspected of containing a microorganism (e.g. a pathogenic or disease-causing microorganism of any type).

In some embodiments, the fluid sample is an environmental sample. The term “environmental sample” refers to any sample obtained from the environment. Common environmental samples include air samples, water samples, soil samples, samples of biological materials, and samples of wastes (liquids, solids or sludges). For example, an environmental sample may be a sewage, soil, water, or air sample. Environmental samples also include sampling of inanimate objects and surfaces (e.g. samples and swab samples taken from buildings, etc.) and the sampling and swab sampling of the inanimate environment in facilities (e.g. healthcare facilities) including environmental surfaces such as floors, walls, equipment and instruments, furniture, and other parts of the physical infrastructure, including air filters, HEPA filters, water filters, and other filters.

The term “sufficiently miscible” as used herein refers to the capacity of two fluids to interact with each other to remove any significant barrier to the transfer of the target from one fluid to the other (e.g. a fluid-fluid interface barrier, such as an oil-aqueous fluid interface). For example, a fluid sample that is sufficiently miscible with a destination fluid allows for transfer of the target from the fluid sample to the destination fluid. Sufficiently miscible does not necessarily imply that the two fluids will become a homogenous mixture. Rather, in some instances the two fluids may remain mostly separate but still interact with each other sufficiently or otherwise permit movement of the target from one fluid to the other. For example, layers in a sucrose density gradient are sufficiently miscible. Likewise, a biological sample containing the target may be sufficiently miscible with a wash buffer or with an elution buffer, thereby permitting movement of the target from the biological sample to the buffer. As a counter example, although air is partially miscible with water, it is not sufficiently-miscible to facilitate interaction at the air-water interface to permit transfer of particles. Accordingly, air and water would not be referred to herein as “sufficiently miscible”. Likewise, substances such as silicone oil and water also do not create a sufficiently miscible interface to facilitate transfer from a container comprising a sample (e.g. complete or near-complete target-PMP complex transfer). In some embodiments, the methods and systems of the invention provide for complete target transfer (including, for example, complete target-PMP complex transfer) from a sample or container comprising a sample to a destination fluid or position within a destination container. In some embodiments, the methods and systems of the invention provide for complete target-contaminant transfer (including, for example, complete contaminant-PMP complex transfer) from a sample or container comprising a sample to a destination fluid or position within a destination container.

The term “subject” as used herein refers to an entity from which a sample is obtained. The subject may be a mammal. In some embodiments, the subject is a human. In some embodiments, the subject is a plant. In some embodiments, the subject is an inanimate object.

The term “target” as used herein is used in the broadest sense and refers to any desired material that is to be isolated from and/or detected in a sample. In some embodiments, the target binds to a paramagnetic particle and is isolated from the sample via application of a magnetic force. In other embodiments, the target is a particle of sufficient density to be isolated from the sample without application of a magnetic force (e.g. via gravity), or via application of another force (e.g. centrifugation, etc.) For example, the target could be a cell, which can be isolated via gravity and/or centrifugation. In some embodiments, the target is a protein (e.g. antibody), whole cell, or a nucleic acid (e.g. DNA, RNA). In some embodiments, the target is a metabolite, a carbohydrate, a glycopeptide, or a lipid.

The terms “analyte” or “target analyte” refer to any substance that is to be, or is being, extracted, isolated, identified or measured.

### DETAILED DESCRIPTION

In some aspects, provided herein are systems and methods for extracting and/or isolating at least one target from a fluid sample. In some embodiments, provided herein are systems and methods for isolating at least one target from a biological sample.

The methods described herein possess many advantages over currently employed sample purification technologies and technologies for extracting and/or isolating at least one target from a sample (e.g. a biological sample). Current tip-based sample purification technologies rely on the use of fluid and air interfaces or fluid and oil interfaces or immiscible fluid interfaces as “barriers” to limit contaminant carryover. These processes can be difficult and fastidious, requiring precise distances, magnetic field strengths, and immiscible fluids such as oil. Likewise, these barriers create a fundamental issue for accomplishing transfer of 100% of all particles. In contrast, the methods described herein rely on the creation of passable restrictions, which permit transfer of the target from a sample to a destination fluid while minimizing contamination.

The methods described herein rely on the use of various techniques to allow passage of desired particles (e.g. a target or analyte) from the sample to a destination fluid, while reducing the passage of unwanted particles (e.g. contaminants). The terms “particle” and “particles” as used herein is meant to be inclusive of both “target” particles (e.g. a target or analyte) and “contaminant” particles (e.g. contaminants). The terms “contaminants” or “contaminant particles” are used interchangeably in the broadest sense and comprise solids (e.g. unwanted nucleic acids, proteins, cell debris, etc.) and/or liquids (e.g. buffers, sample fluid, etc.). In some embodiments, allowing passage of the desired particles may comprise allowing passage of the target particle into the destination fluid. In some embodiments, reducing passage of contaminant particles may comprise reducing passage of a liquid (e.g. the liquid sample using prewash-aspiration and/or postwash-aspiration, Bubble-It, reduced interfacial area or interaction time between fluids, etc.) into the destination fluid, while the target particles themselves are able to pass into the destination fluid. In other embodiments, allowing passage of the desired particles comprises allowing passage of contaminants into the destination fluid, while reducing passage of the desired particle (e.g. the target particle) into the destination fluid.

In some embodiments, the methods rely on the passage of the desired target from the sample to the destination fluid, while preventing/minimizing passage of contaminants into the destination fluid. In some embodiments, passage of contaminants is restricted by restricting the flow of a liquid containing the potential contaminants. For example, in some embodiments, preventing/minimizing passage of contaminants comprises permitting passage of the desired target (e.g. the desired target particle) contained within a liquid sample to the destination fluid, while restricting the flow of the liquid sample itself. Thus, the method restricts the flow of the liquid containing potential contaminants, thereby minimizing passage of contaminants into the destination fluid.

In some embodiments, the methods described herein involve the passage of contaminants from the sample to the destination fluid. Although the methods described herein include the target passing from the sample to the destination fluid (e.g. positive selection), it is understood that the methods and techniques described herein may also be designed such that contaminants pass from the sample to the destination fluid, leaving the desired target behind (e.g. negative selection). In both positive and negative selection, the target is considered to be “isolated” from the fluid sample in that the target is substantially separated from potential contaminants. As used herein, the term “isolated” or “isolating” or “purified” or “purifying” refer to the act of separating the target from one or more or all contaminants. The terms are meant to include methods of isolating involving the transfer of the target from the sample to the destination fluid (e.g. positive selection of the target) and methods of isolating involving retaining the target within the container while contaminants pass from the sample to the destination fluid (e.g. negative selection).

In some embodiments, the methods comprise placing a container housing the sample into a destination fluid. In some embodiments, the methods comprise placing a container housing the sample onto a surface. In some embodiments, placing the container housing the sample onto the surface results in transfer of an amount of fluid from the container to the surface, thereby generating the destination fluid on the surface. In some embodiments, the surface comprises a hydrophobic material. In other embodiments, the surface comprises a hydrophilic material. The surface may be glass or plastic, such as a well or a dish. In other embodiments, the surface may comprise porous material. The porous material may be wet or dry. In other embodiments, the surface may comprise a magnet. In other embodiments, the surface may comprise an assay material (e.g. lateral flow assay sample pad, etc.). In some embodiments, the surface comprises a pad (e.g. an absorbent pad, such as a cotton pad, a blood spotting disc, etc.). In some embodiments, the container housing the sample is brought into proximity with a surface and a small volume of fluid is dispensed, passively or actively, from the container to generate a destination fluid on the surface. Such a method can be advantageous when the surface is rigid and/or impact of the container with the surface is undesirable. In some embodiments, after generation of destination fluid, an amount of destination fluid may be aspirated back into the sample container to minimize carryover of background contaminants.

In some embodiments, the container comprises an opening that permits movement of particles from the sample into the destination fluid. In some embodiments, the particles are target particles. In some embodiments, the particles are contaminant particles. In some embodiments, the container comprises an opening that permits movement of potential contaminants from the sample into the destination fluid, thereby leaving the target within the initial container. In some embodiments, the sample is sufficiently miscible with the destination fluid. Accordingly, movement of the target or movement of potential contaminants from the sample into the destination fluid is able to occur.

The systems and methods described herein may be used for isolation of any desired target from the sample. For example, the target may be a nucleic acid (e.g. DNA, RNA, or various subtypes thereof including mRNA) a protein, a metabolite, a carbohydrate, a glycopeptide, or a lipid. In some embodiments, the target may be nucleic acid or proteins (e.g. antibodies) resulting from a pathogen infecting the subject from which the biological sample was obtained. For example, the target may be bacterial nucleic acid (e.g. bacterial DNA or RNA) or viral nucleic acid (e.g. viral DNA or RNA). As another example, the target may be antibodies produced by the subject in response to infection with the pathogen. In some embodiments, the target may be a cell.

In some embodiments, the particle (e.g. the target or the contaminant) is bound to a capture moiety. In some embodiments, the target is bound to at least one capture moiety. In some embodiments, the contaminant is bound to at least one capture moiety. In some embodiments, the capture moiety is a paramagnetic particle (PMP). In some embodiments, the particle is bound to a capture moiety to form a complex. For example, in some embodiments the particle is bound to a PMP to form a particle-PMP complex. The particle-PMP complex may be a target-PMP complex or a contaminant-PMP complex. In some embodiments, the target is bound to at least one paramagnetic particle (PMP) to form one or more target-PMP complexes. In some embodiments, the methods comprise mixing the fluid sample (e.g. a biological sample) with the capture moiety (e.g. PMPs). Mixing the sample with PMPs allows the PMPs to bind to the particle in the sample, thus generating one or more complexes. For example, in some embodiments mixing the sample with PMPs allows the PMPs to bind to the target, if present in the sample, thus generating one or more target-PMP complexes.

The PMPs may be contained in a liquid formulation. Alternatively, the PMPs may be in a lyophilized formulation. Lyophilized PMP formulations may contain other suitable reagents commonly used in the lyophilization process, including bulking agents, stabilizers, and other suitable excipients. For example, lyophilized PMPs may be present in the container (e.g. pipette tip) housing the fluid sample. As another example, PMPs may be added to the sample, either before aspirating the sample-PMP mixture into the container, or after the sample is present in the container.

Any suitable paramagnetic particle may be used. In some embodiments, paramagnetic particles may be purchased from a commercial vendor. The specific type of paramagnetic particle used depends on the target to be isolated from the biological sample. For example, particles with a relatively large surface area may be preferable for binding nucleic acid, such as viral RNA. In some embodiments, the paramagnetic particles may be functionalized to aid in capture/purification of the target. For example, the paramagnetic particles may be functionalized with one or more antibodies, aptamers, or other suitable agents to assist with capture of a target. In some embodiments, the paramagnetic particles may be functionalized with one or more spike protein antibodies to assist with the capture of SARS, coronavirus, SARS-CoV-2 and related targets. In some embodiments, the paramagnetic particles may be functionalized with a suitable moiety to facilitate capture of target antibodies. For example, the paramagnetic particles may be functionalized with a spike protein or synthetic variant thereof to facilitate capture of antibodies produced as a result of infection with SARS, coronavirus, SARS-CoV-2, or other related pathogens.

Any suitable amount of PMPs may be present in the sample. In embodiments where the PMPs are contained in a liquid, any suitable volume of the liquid composition comprising paramagnetic particles may be present in the sample. Any suitable concentration of PMPs may be used to ensure sufficient binding of the PMPs to the target (e.g. formation of a sufficient number of target-PMP complexes). For lyophilized PMP formulations, any suitable weight of lyophilized product may be used to ensure the proper concentration of PMPs are present within the fluid sample. For liquid formulations, the liquid composition comprising the PMPs may comprise any suitable concentration of PMPs to ensure sufficient binding of the PMPs to the target (e.g. formation of a sufficient number of target-PMP complexes). A suitable amount of PMPs may depend on many factors, including the volume of the liquid sample, the size of the beads, the intended target to be isolated, and the like. For example, PMPs may be present at about 0.01-98% (v/v). For example, PMPs may be present in an amount of about 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, about 20%, about 25%, about 30%, about 35%, about 40%, about 45%, about 50%, about 55%, about 60%, about 65%, about 70%, about 75%, about 80%, about 85%, about 90%, about 95%, or about 98% (v/v).

In some embodiments, the fluid sample contains other suitable reagents for processing/handling of samples. For example, the fluid sample may contain one or more detergents, reducing agents, buffers, inhibitors, enzymes (e.g. proteases), denaturants, etc. For example, the sample may further comprise one or more reagents to decrease viscosity of the sample. For example, the sample may comprise PMPs and DTT. The sample may comprise other suitable buffers, inhibitors, and the like to prevent degradation of the target (e.g. target nucleic acid, target protein, etc.) during sample processing. Suitable inhibitors that may be present in the sample include, for example, RNase inhibitors, protease inhibitors, nuclease inhibitors, and the like.

The destination fluid may be any suitable fluid, including the same or similar fluid that comprises a sample. In some embodiments, the methods described herein are used with the goal of transferring a target of interest from the sample to the destination fluid. In other embodiments, the target may be a contaminant, and the methods described herein are used with the goal of transferring contaminants from the sample, while leaving behind a target of interest (e.g. a protein, a nucleic acid, etc.) within the container. In any of the methods described herein, the destination fluid may be a liquid. For example, the destination fluid may be a wash buffer. As another example, the destination fluid may be an elution buffer. In some embodiments, the destination fluid comprises water. In some embodiments, the destination fluid comprises ethanol. In some embodiments, the destination fluid comprises substantially the same fluid material as in the fluid sample (including, e.g., in embodiments where the destination fluid is generated on a surface by placing the container housing the sample fluid onto the surface or by dispensing some of the fluid sample onto a surface).

The fluid sample and the destination fluid may be any suitable fluids that are sufficiently miscible with one another, such that passage of the target from the sample to the destination fluid can occur or such that passage of contaminants from the sample to the destination fluid can occur. In some embodiments, passage of particles (e.g. passage of the target, or passage of the contaminant) occurs through diffusion. In some embodiments, passage of particles occurs due to gravity or other form of acceleration (e.g., via a centrifuge). In some embodiments, movement of a particle-PMP complex is induced. For example, in some embodiments movement of a particle-PMP complex is induced, such as through application of a magnetic force to the system. For example, a magnetic force may be applied, thereby drawing the particle-PMP complexes through the opening and into the destination fluid. As another example, in some embodiments the container housing the sample is placed on a surface and the destination fluid is generated on the surface. Particle-PMP complexes may aggregate at the interface of the container and the surface, and a magnetic force may be applied to hold the beads on the surface, while the container is removed. Accordingly, the target-PMP complexes are transferred from the container to the destination fluid that has been generated on the surface. In some embodiments, such a process of bringing the container into contact with a surface may be performed multiple times without refilling the container with sample, thereby generating multiple spots of target-PMP complexes in series. In some embodiments, the method of isolating a target from a fluid sample comprises placing a container housing the sample comprising one or more target-PMP complexes into a destination fluid, and applying a magnetic force to draw the target-PMP complexes from the sample into the destination fluid. In other embodiments, the method comprises placing a container housing the sample comprising one or more contaminant-PMP complexes into a destination fluid and applying a magnetic force to draw the contaminant-PMP complexes from the sample into the destination fluid.

The systems and methods described herein rely on the use of passable restrictions to facilitate target purification while minimizing contamination. As used herein, the term “passable restriction” refers to any suitable mechanism which permits movement (e.g. passage) of the desired particle (e.g. passage of the target, or passage of contaminants) from the fluid sample to the destination fluid, while also restricting (e.g. limiting) the passage of unwanted particles from the fluid sample to the destination fluid. For example, in some embodiments a “passable restriction” refers to a suitable mechanism including those described above which permit movement of the target from the fluid sample to the destination fluid, while minimizing the amount of contamination that occurs before, during, and/or after said movement of the target. This process is referred to herein as “positive selection”. In other embodiments, a “passable restriction” refers to a mechanism which permits passage of contaminants from the fluid sample to the destination fluid, while the desired target remains within the container holding the fluid sample. This process is referred to herein as “negative selection”.

The claimed systems and methods are designed to employ passable restrictions to minimize contaminations that would otherwise occur during transfer between fluids. Undesirable contamination can occur in various ways. In the context of the claimed method, contamination generally occurs by (a) contaminated surface carryover, (b) diffusion, (c) convection (e.g., via pressure, differences in density, or temperature differences), (d) settling (e.g., background cells settling downward during isolation of target cells), (e) via association with beads (e.g., on the surface or in the bulk via direct or indirect connection), or (f) as the fluid in the tip touches the wash solution, from surface-tension-based forces (e.g., Marangoni). Typically, contamination from surface carryover, diffusion, and convection dominate. Accordingly, various passable restrictions may be employed to reduce contamination, such as contamination from any of the above-mentioned sources.

In some embodiments, at least one passable restriction is employed to minimize diffusion-based contamination. Diffusion-based carryover is influenced by multiple parameters, including: (1) geometry (e.g., cross-sectional area of the opening), (2) diffusion coefficients, and (3) time, any or all of which may be manipulated, set or adjusted in the methods and systems of the invention, as desired.

In some embodiments, at least one passable restriction is employed to minimize convection-based contamination. Convection carryover is essentially any non-diffusion-based movement of the fluid and can result from many different causes including: (1) differences in density between fluids (e.g., as induced by temperature or salinity) which, in the presence of gravity/acceleration, can induce convection, (2) momentum (e.g., movement or vibration of the container), (3) pressure (e.g., applied by the pipette pressure mechanism), (4) and entrainment (e.g., movement of bodies within the fluid that induces convection).

In some embodiments, a passable restriction is achieved by control of the size of magnetic particle used to capture a target analyte and/or a target contaminant.

In some embodiments, a passable restriction is achieved by control of the size and/or nature of the magnetic particle used to capture a target analyte and/or a target contaminant. In some embodiments, for example, a passable restriction is achieved by use of a ferromagnetic particle for the capture and movement of one target- or contaminant-complex and the use of a paramagnetic particle for the capture and movement of another target- or contaminant-complex. A passable restriction can also be created by control of the size of a magnetic particle used to capture a target analyte and/or a target contaminant (e.g. the size of the ferromagnetic particle and/or paramagnetic particle).

In some embodiments, a passable restriction is achieved by control of the geometry of the container for housing a sample and the opening thereof. For example, a passable restriction may be achieved by controlling the size and/or shape of the container and the size and/or shape of the opening of the container. The container may be any suitable container comprising an opening, including but not limited to pipette tips, tubes, capillary tubes, straws, etc. Generally speaking, a larger opening (e.g. a larger pipette tip orifice) allows for more diffusion to occur in the same unit of time compared to diffusion for a smaller opening. Accordingly, a larger opening permits an increased passage of potential contaminants into the container (e.g. into the pipette tip) compared to a smaller opening. In other words, a larger opening permits increased diffusion-based contamination compared to a smaller opening. However, a large opening also facilitates transfer of the target out of the container. For example, a larger opening can help avoid blockages that may otherwise occur during transfer of the target out of a smaller pipette tip. Accordingly, in some embodiments a passable restriction to minimize diffusion-based contamination comprises selecting an appropriately sized opening to facilitate appropriate transfer of the target out of the container, while minimizing the number of potential contaminants that may leave the opening by diffusion into the destination fluid.

In some embodiments, the opening is roughly circular. In some embodiments, the opening is roughly circular with an average inside diameter of about 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, or 2.5 mm, 2.6 mm, 2.7 mm, 2.8 mm, 2.9 mm, 3.0 mm. In some embodiments, larger openings may be needed. For example, for a sewage sample containing flocculated (e.g. clumped) contaminants, the diameter of the opening may be more than 3.0 mm. For example, the diameter may be 3.0 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, or larger.

In some embodiments, the container comprises more than one opening. For example, the container may comprise an array of openings. Such an array of openings may provide many small areas where flux or flow can be achieved. For example, such an array of openings provides many small areas where a target (or a contaminant, in the case of negative selection) can pass through the openings, yet viscous resistance increases roughly proportional to 1/r\hat{ }4 where r is the radius of the opening. Thus, use of many small holes strongly resists unintended convective transport of contaminants yet allows passage of target, even if one or more other openings become blocked. In contrast, if only a single opening is used, convective contamination is much easier and blockage will prevent any subsequent transfer.

In some embodiments, the opening may be brought into proximity with a surface of a container housing the destination liquid. For example, the opening may be brought into contact with a surface of a container housing the destination liquid, effectively reducing the cross-sectional area through which diffusion or convection would otherwise occur. In some embodiments, the opening may be brought into contact with a surface of a container housing the destination, thereby closing off the container opening (e.g. pipette tip), thereby preventing diffusion of contaminants through the opening, while the target aggregates proximal to the opening within the container housing the sample. After sufficient aggregation of the target occurs, the container opening (e.g. pipette tip) may be removed from the destination liquid, thereby leaving behind the target within the destination liquid while minimizing diffusion-based carryover. In other embodiments, the opening may be brought into contact with a surface of a container housing the destination, thereby closing off the container opening (e.g. pipette tip), thereby preventing diffusion of the target through the container opening, while the contaminants aggregate proximal to the opening within the container housing the sample. After sufficient aggregation of contaminants occurs, the container (e.g. pipette and/or pipette tip) may be removed from the destination liquid, thereby depositing the contaminants within the destination liquid while retaining the target within the pipette tip (e.g. within the container).

In some embodiments, a passable restriction is achieved by using pressure. For example, in some embodiments pressure is applied to induce convection of fluid into the opening (e.g., by aspirating fluid with the container), thereby causing flow in the opposite direction of the analyte (e.g. the target, or the contaminants) being transferred (i.e., retrograde flow). Retrograde flow before, during, or after transfer can prevent diffusion-based contamination. Retrograde flow may be used to prevent unwanted passage contaminants if the target is the analyte being transferred. In other embodiments, retrograde flow may be used to prevent unwanted passage of the target out of the container if a negative selection process is being employed.

The Peclet number is used to describe the relative balance of convection/advection and diffusion. As used herein, the term “convection” applies to movement of a fluid, whereas “advection” refers to movement of particles dissolved or suspended within the fluid. When the Peclet number is 1, diffusion and convection/advection are approximately balanced. When larger than 1, convection/advection dominates diffusion, preventing contamination. Movement within that flow (e.g. movement of the target) may be influenced by outside forces such as acceleration/gravity or magnetic force, which is not accounted for in the Peclet number. Thus, in this example, the Peclet number refers to the transport of contaminants and movement of the contaminant is essentially dictated by the flow of the fluid and diffusion of the contaminant within that fluid. With sufficient retrograde flow (e.g., Pe>>1), diffusion-based movement of contaminants out of the container (e.g. a pipette or pipette tip), into the destination fluid, is prevented while external forces allow the target to be transferred despite the retrograde flow. However if fluid flow is too fast, drag forces on the target in transfer could prevent the target (e.g. target particles) from transferring. The fluid velocity at the tip that would prevent transfer can be approximated using the Stokes equation for the drag force on a particle in flow that would balance out any forces that are aiding transfer (e.g., gravity or magnetism).

In some embodiments, the method comprises aspirating fluid into the container after the container is placed into the destination fluid. In some embodiments, fluid is aspirated into the container before, during, and/or after movement (e.g. movement of the target, or movement of contaminants, or both) from the container to the destination fluid. In some embodiments, fluid is aspirated into the container prior to transferring the desired entity (e.g. the target and/or the contaminants). Aspirating first pulls the fluid into the container, such that any potential particles traveling by diffusion must first travel through the aspirated portion of the fluid before they can escape. Aspirating fluid such that multiple millimeters of fluid are in the container (e.g. pipette tip) can provide many minutes of time before significant diffusion-based transfer of particles (i.e., target or contaminants) can occur from the sample within the container to the destination fluid. In some embodiments, diffusion-based contamination can be estimated using the equation:

$$t \approx L \hat{} 2 / 2 D$$

where t=time, L is the number of millimeters of fluid aspirated into the container, and D is the diffusion coefficient of the potential contaminants within the fluid sample.

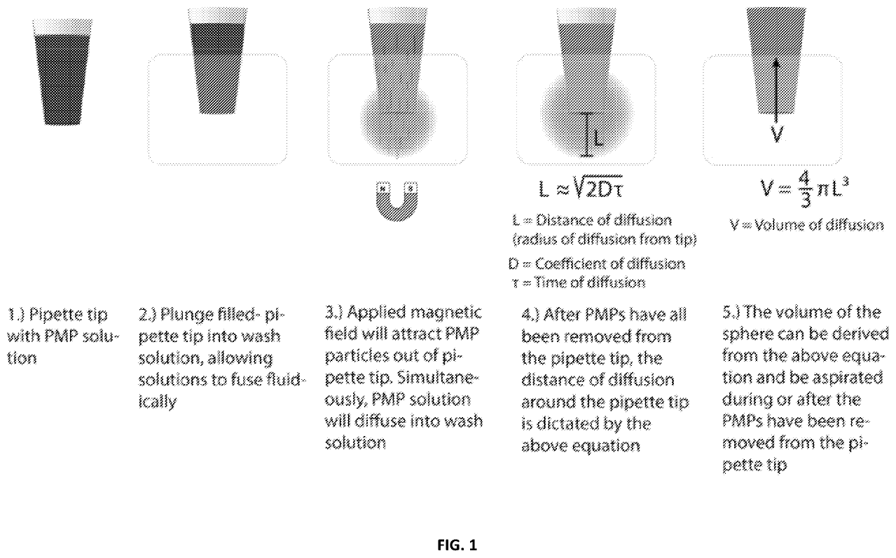

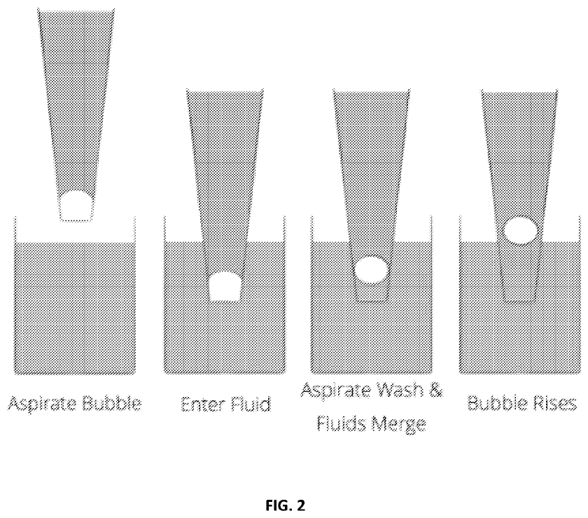

In some embodiments, staggering the timing of the drag and transfer forces simplifies operation and provides robust performance. In some embodiments, the method comprises aspirating fluid into the container following transfer (e.g. transfer of the target, transfer of contaminants, or both) from the sample to the destination fluid. Aspiration after transfer may be performed to recoup potential contaminants that carried over (see FIG. 1). Contaminants that carried over via diffusion will diffuse slowly and the distance they diffuse can be estimated conservatively using the following equation based in 1-dimension (vs. a potentially more accurate 3D model):

$$L = sqrt ( 2 * D * t )$$

where L is the diffusion distance, D is the diffusion coefficient, and t is the time that diffusion has been allowed to proceed (see FIG. 1, step 4). This distance then informs how much of the volume surrounding the tip should be aspirated to recoup the diffusion-based contaminants. For example, volume can be calculated using the equation

$$v = 4 / 3 * pi * {L}^{3}$$

where v is the volume of diffusion, and L is the diffusion distance.

In some embodiments, aspiration may occur both before (e.g. pre-aspiration) and after (e.g. post-aspiration) transfer of the target. In some embodiments, pre- and post-aspiration can both be performed to provide superior protection against contamination.

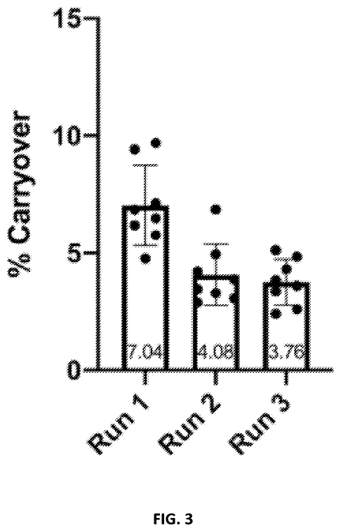

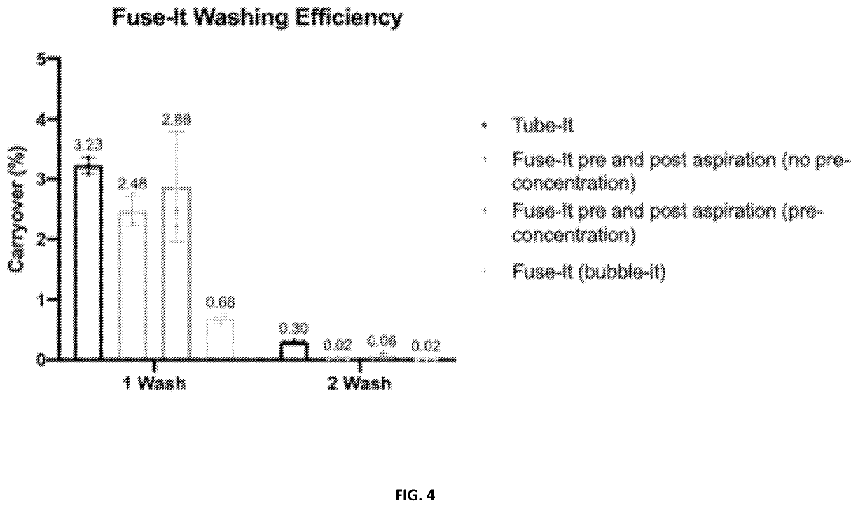

In some embodiments, a passable restriction is achieved by generating a pocket of air, oil, or gas proximal to the opening of the container. In some embodiments, a passable restriction is achieved by generating a pocket of air (e.g. an “air bubble”) or oil (e.g. an “oil bubble”) proximal to the opening of the container prior to inserting the container into the destination fluid. In some embodiments, a passable restriction is achieved by generating an air pocket proximal to the opening of the container prior to inserting the container into the destination fluid. Such an embodiment is shown in FIG. 3. In some embodiments, an air pocket may be preferred over or advantageous compared to an oil pocket. For example, when using standard pipette tips as the container air may be advantageous because the standard polypropylene pipette tip material is lipophilic. Being lipophilic, pre-aspiration with oil would create a barrier that interacts strongly with the pipette, creating a barrier to the transfer of the target. In other words, the use of oil in such an embodiment may represent a non-passable restriction. In contrast, air has a weaker interaction than oil with the polypropylene, allowing the air bubble to separate from the pipette wall more easily and enable the fluids to merge around the sides of the bubble. This can be enhanced with surface treatments or sample/wash additives (e.g., for aqueous solutions, precoating with surfactant such as BSA or detergent or via oxygen plasma treatment as well as detergents or surfactants in sample or wash solutions) that enhance interaction between the sample and the pipette tip.

In some embodiments, the method for isolating a target from a fluid sample comprises generating a pocket of air proximal to the opening of the container, inserting the container into the destination solution, and aspirating fluid into the container. In such embodiments, aspirating fluid in the container forces the air pocket (e.g. bubble) to rise within the container with the air bubble initially positioned at the interface of the two fluids, thereby permitting contact between the fluid sample and the destination fluid yet restricting the area of the initial interface between the two fluids. Following contact of the two fluids, transfer of the target from the sample to the destination fluid can occur around the bubble, through the sufficiently miscible interface between the two fluids. Alternatively, transfer of contaminants from the sample to the destination fluid can occur around the bubble, while the target remains held within the container.

In some embodiments, the wall of the container (e.g. pipette) is tapered; therefore, as fluid is aspirated and the air moves upward, it forms a bubble that eventually becomes a sufficiently spherical to allow the fluids to merge around the bubble circumference depending on the relative philicity/phobicity of the sample/wash fluid, tip material, and what is used for the “bubble” (in some embodiments it is air, for example, but it could also be a hydrophobic liquid such as oil or a specialized gas). The volume of air should be optimized to facilitate appropriate transfer of the desired moiety from the sample to the destination fluid. For instance, if too much air is aspirated the bubble becomes roughly cylindrical in shape separating the fluids by a large distance and preventing fusion or interfacing of the two fluids. In contrast, if too little air is aspirated, the area of the interface between the two fluids is not significantly reduced for preventing contamination. When performed successfully, the bubble either stays in the container “in between” the two fluids (essentially stuck to one side of the pipette wall), or the bubble will dislodge and rise. In either case, mixing of the fluids during fusion is contained within the pipette tip and a diffusion/convection barrier is established, and beads are allowed to pass through wash fluid in the pipette tip before entering the destination fluid.

The volume of the air pocket may vary depending on the geometry of the container and/or the opening of the container. In addition, the volume of the air pocket may vary depending on whether the interior surface of the container is treated with a suitable agent/additive. In addition, the volume of the air pocket may vary depending on whether the sample fluid comprises additional agents, such as detergents, that may influence interactions between the sample and the container.

In some embodiments, the volume of the air pocket is between about 1 μl and about 100 μl. In some embodiments, the volume of the air pocket is about 1 μl, about 5 μl, about 10 μl, about 15 μl, about 20 μl, about 25 μl, about 30 μl, about 35 μl, about 40 μl, about 45 μl, about 50 μl, about 55 μl, or about 60 μl. In some embodiments, the volume of the air pocket depends on the dimensions of the container (e.g. pipette tip) holding the air pocket. For example, in some embodiments, the air pocket is of sufficient volume such that the distance from the container (e.g. pipette tip) opening to the top of the air pocket (e.g. the portion of the air pocket farthest away from the pipette tip opening) is roughly equivalent to the diameter of the container (e.g. pipette tip). In some embodiments, the air pocket is of sufficient volume such that the distance from the container (e.g. pipette tip) opening to the top of the air pocket is about 50% to about 300% of the diameter of the container (e.g. pipette tip) opening (e.g. about ½ the diameter of the opening to about 3 times the diameter of the container (e.g. pipette tip) opening). For example, the distance may be about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% of the diameter of the container (e.g. pipette tip) opening.

In some embodiments, interaction between the fluid sample and the destination fluid (e.g. fusion) can be initiated or assisted by adding a surfactant to the fluid sample. For example, addition of a surfactant may help facilitate interaction between the fluid sample and the destination fluid when an air pocket has been generated within the container. As another example, interaction between the fluid sample and the destination fluid may be initiated or assisted by inducing motion within the container, in particular in aspects where an air pocket (or an oil pocket, or a gas pocket) has been generated. For instance, flicking or otherwise disturbing the container can induce motion of the air pocket, thereby allowing fusion of the fluid sample and the destination fluid to occur within the container.

In some embodiments, the geometry of the container may be selected to optimize the use of the air, oil, or gas pocket as a passable restriction. For instance, the geometry of the container may be selected to help maintain the integrity of the air pocket during fluid fusion (e.g. during interaction of the fluid sample with the destination fluid). In some embodiments, a crenellated ridge on the inner surface of the pipette tip forming a ring shape can help create and maintain a more effective passable restriction. For instance, this radial restriction will help prevent a bubble from rising within the pipette tip after fluid fusion, thus allowing the bubble restriction to be maintained during bead transfer. Alternatively, surface patterning or geometry may be used to create preferential pinning points within the pipette, such that multiple fluids could be aspirated with a bubble between them. For instance, a pinning point could be created such that a first wash fluid and a second wash fluid can be aspirated. When additional wash fluid is aspirated, the conical nature of the container (e.g. pipette tip) would induce a larger vertical displacement of the lower interface than the upper interface, causing them the first to break free of the pinned location first, bringing the wash solution into communication (e.g., fuseing it) with the sample fluid.

In some embodiments, at least one technique is employed to reduce carryover contamination from a contaminated surface. For example, at least one technique is employed to reduce potential contamination from the surface of the container when placed into the destination fluid. For example, in some embodiments the method of isolating a target comprises placing a portion of the container (e.g. pipette tip) into a cleansing fluid to remove contaminants from an exterior surface of the container. For example, a portion of the container proximal to the opening of the container may be placed into a cleansing fluid to remove contaminants from the exterior surface of the container prior to placing the container within the destination fluid. Such a step may be referred to herein as a “pre-rinse” of the container. In the case of a pipette tip, for example, a portion of the pipette tip may be placed into a cleansing fluid prior to placing the pipette tip into the destination fluid and may be referred to as a “Tip-Dip.” Placement into the cleansing fluid thereby minimizes the passage of potential contaminants from the outside surface of the container (e.g. pipette tip) into the destination fluid (e.g. when the pipette tip is placed into the destination fluid).

Another suitable technique that may be employed to minimize carryover contamination is to restrict the surface area in contact with the sample fluid. This can be done in multiple ways. One way to do this is to follow the level of the sample fluid during aspiration into the container. In liquid handling terminology, this might be referred to as “liquid following”. This provides a way to aspirate a fluid down to the bottom of a container without having to unnecessarily submerge the pipette tip into the sample. Another way would be to use microstructures on the container (e.g. pipette tip). For example, sharp edged grooves could be added along the length of a hydrophobic pipette tip. Upon submerging into an aqueous sample, sample will avoid wetting inside the grooves, thereby reducing the wetted/contaminated surface area and thus carryover of surface contaminants. Nanostructures or textures used to create superhydrophobic surfaces function similarly and would reduce surface-based carryover contamination. Accordingly, such methods can also be viewed as passable restrictions that permit transfer of the target while minimizing potential carryover contamination from the surface of the container (e.g. the surface of the pipette tip).

In some embodiments, multiple techniques to reduce contamination are employed. For example, in some embodiments a pre-rinse step and at least one passable restriction are employed. In some embodiments, multiple passable restrictions are employed. For example, the method may comprise generating multiple passable restrictions to minimize contamination, including any combination of the above-described passable restrictions.

In some embodiments, the methods further comprise pre-concentrating the target analyte proximal to the opening of the container. For example, pre-concentrating may be performed by applying a first magnetic force to the container to generate a concentration of target-PMP complexes proximal to the opening of the container. Following generating the concentration of target-PMP complexes proximal to the opening of container, the container may be placed into the destination solution and the target may be transferred from the container to the destination fluid, such as by diffusion or by application of a magnetic force to draw the target-PMP complexes into the destination fluid. In such embodiments, the aggregation of target-PMP complexes themselves serves as a restriction to the passage of potential contaminants. For example, the aggregation of target-PMP complexes may serve as a physical blockage that minimizes passage of potential contaminants from the pipette tip into the destination fluid, as these contaminants are trapped above the complexes (e.g. trapped in the pipette tip, farther away from the destination fluid compared to the complexes).

In other embodiments, the destination fluid may be housed within a suitable container that further comprises a cleansing fluid. For example, the destination fluid and the cleansing fluid may exist as separate layers, such that the cleansing fluid is on top of the destination fluid. The container therefore passes through the cleansing fluid prior to contacting the destination fluid, thereby facilitating removal of potential contaminants on the exterior surface of the container by the cleansing fluid prior to contact with the destination fluid. Suitable cleansing fluids include, for example, liquids or oils. For example, the cleansing fluid may be a liquid of lower density than the destination fluid. As another example, the cleansing fluid may be a hydrophobic liquid. For example, the cleansing fluid may be a layer of oil, such as mineral oil, that floats on top of the destination fluid. In some embodiments, the cleansing fluid and the destination fluid are not sufficiently miscible, thereby preventing diffusion of the contaminants from the cleansing fluid to the destination fluid. For example, oil and water are not sufficiently miscible and thereby represent effective cleansing fluids (e.g. oil) and destination fluid (e.g. water), respectively. In some embodiments, the cleansing fluid (e.g. oil) acts as a barrier (e.g. passable restriction) that pushes back residual fluid/contaminants on the outside of the pipette tip as the tip is dipped through the oil. For example, as the pipette tip is dipped through the oil layer and submerged into the destination fluid, the oil layer pushes potential contaminants on the outside of the pipette tip up the pipette tip, thereby preventing these carryover contaminants from contacting the destination fluid.

The systems and methods described herein may be used to isolate a target from any suitable fluid sample (e.g. a target analyte and/or target contaminant). In some embodiments, the sample is an environmental sample. In some embodiments, the sample is a biological sample. The sample may be collected and/or stored in a suitable container (e.g. a sample collection container) prior to processing the samples by the methods described herein. Any type of sample collection container may be used that is suitable for receiving a sample and storing the sample until performing the described methods for detection of the target. Examples of sample collection containers include, but are not limited to, tubes containing a reversibly removal cap, bags, syringes, droppers, and the like. In some embodiments, the samples are pre-treated prior to performing the methods described herein.

In some embodiments, the sample may be pre-treated to inactivate potential pathogens (e.g. virus, bacteria) within the sample. In some embodiments, the sample may be pre-treated to lyse cells within the sample, thus releasing the target (e.g. nucleic acid) for subsequent isolation. In such embodiments, a pre-treatment step accomplishes both cell lysis (e.g. release of nucleic acid) and inactivation of potential pathogens within the sample. In some embodiments, the samples may be pre-treated by freezing, heating and/or the addition of a denaturant to the sample. For example, the sample may be pre-treated by heating to a sufficient temperature for a suitable duration of time to inactivate potential pathogens within the sample. For example, the sample may be heated to about 40° C. or higher. For example, the sample may be heated to about 40° C., 45° C., 50° C., 55° C., 60° C., 65° C., 70° C., 75° C., 80° C., 85° C., 90° C., 95° C., 100° C., or more than 100° C. The sample may be maintained at the heated temperature for a suitable duration of time, such as 5 minutes, 10 minutes, 20 minutes, 30 minutes, 40 minutes, 50 minutes, 60 minutes, or more than 1 hour. In particular embodiments, the sample may be heated to 98° C.-100° C. for 5-10 minutes to accomplish both cell lysis and viral inactivation in a single heat treatment step. In some embodiments, pre-treating the sample comprises adding a denaturant to inactivate potential pathogens within the sample. For example, suitable denaturants include guanidine-based denaturants (e.g. guanidine hydrochloride, guanidine thiocyanate, etc.) and surfactants (e.g., Triton X-100, tween20).

The sample may additionally comprise a suitable detergent. For example, the sample may comprise an ionic detergent (e.g. sodium dodecyl sulfate, deoxycholate, cholate, etc.), a non-ionic detergent (e.g. Triton X-100, DDM, digitonin, Tween 20, Tween 40, Pluronic F-127), a zwitterionic detergent, or a chaotropic detergent. In some embodiments, the sample comprises 0-5% detergent (v/v). For example, the sample may comprise 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% detergent. In some embodiments, the sample comprises a non-ionic detergent (e.g. Triton X-100). For example, the sample may comprise 0.001-0.1% Triton X-100.

The sample may further comprise a suitable buffer. For example, in some embodiments the sample may comprise phosphate buffered saline (PBS), universal transport medium (UTM), saline, and the like. The sample may comprise one or more enzymes or chemical agents to assist with breaking down the contents therein to facilitate release of the desired target. For example, the sample may comprise one or more enzymes, such as one or more proteases. In particular embodiments, the sample may comprise proteinase K. The sample may additionally comprise one or more suitable reagents to prevent degradation of the target within the sample. For example, suitable buffers and/or inhibitors (e.g. RNase inhibitors, nuclease inhibitors, etc.) may be present in the sample.

In some embodiments, an inner surface of the container housing the fluid sample (e.g. pipette tip) is patterned. In some embodiments, this involves patterning of hydrophilicity/hydrophobicity and/or lipophilicity/lipophobicity. Patterning can be achieved via structural patterning (inclusion/exclusion of edges or boundaries and scaling of features to increase or decrease the dominance of surface tension effects), surface texture (use of micropillars on a hydrophobic surface to make it superhydrophobic), or modification of surface chemistry (e.g., chemical or oxygen plasma treatment of polymers). In some embodiments, such patterning methods are used to help stabilize or encourage positioning of the air pocket (or oil pocket, or gas pocket). Patterning can be used to prevent wetting of surfaces as well for certain container (e.g. tip) designs. In some embodiments, patterning is used to discourage the fluid sample and/or the target contained therein from sticking to the container (e.g. pipette) material.

In some embodiments, the destination fluid is held in a suitable collection device. Any desired collection device or destination container may be used. Examples include, but are not limited to, test tubes, microcentrifuge tubes, dishes, slides, plates, multi-well plates (e.g., 4-well, 8-well, 12-well, 96-well, 384-well, etc.), flasks, vials, channels, and the like. Other suitable examples include porous materials, such as filter paper, and assay surfaces. Drawing a particle or a complex comprising a particle “into” a collection device is meant to encompass any means by which the particle is brought into contact with the sample collection device or a surface thereof. For example, drawing the particle onto a piece of filter paper, onto a plate, etc. is meant to be encompassed by drawing the particle “into” a sample collection device. For automated, high-throughput systems and methods, multi-well plates used in combination with multi-pipette arrays are particularly useful.

In some embodiments, destination fluid held within the collection device comprises a wash buffer. For example, in particular embodiments the distinct wells on a multi-well plate comprise a wash buffer. In some embodiments, the wash buffer comprises water. In some embodiments, the wash buffer comprises ethanol. In some embodiments, a layer of oil (e.g. light mineral oil) may reside above the wash buffer. In some embodiments, the methods described herein further comprises aspirating the destination fluid (e.g. wash buffer) (and potentially the layer of oil, if present) from the sample collection device (e.g. wells) while the device is positioned above the magnet such that the target-PMP complexes remain, and, if desired, allowing the target-PMP complexes to dry. In some embodiments, the target-PMP complexes are not allowed to dry prior to detecting the target. In some embodiments, additional wash steps or overlays with reagents may be performed.

In some embodiments, the destination fluid is removed. In some embodiments, following removal of the destination fluid (e.g. wash buffer) reagents for the detection of the target may be added to the collection device or destination container, and suitable methods for detection of the target may be performed.

In some embodiments, the container, the destination container and/or a sample collection device may comprise reagents for detection of the target.

In some embodiments, reagents for detection of the target are added after removal of some of all of an original destination fluid.

In other embodiments relying on negative selection, the target is retained within the container while potential contaminants pass to the destination fluid. In such embodiments, following removal of contaminants, the target may be brought into contact with a suitable wash buffer, elution buffer, or reagents for detection of the target.

Suitable reagents for detection of the target include, for example, reagents for nucleic acid amplification (e.g. PCR, isothermal amplification, and the like) and/or sequencing. In some embodiments, the reagents for detection of the target comprise reagents for loop-mediated isothermal amplification (LAMP)-based detection of the target. In general, LAMP reactions include a DNA polymerase with strong strand displacement activity and tolerance for elevated temperatures and up to six DNA oligonucleotides of a certain architecture. RT-LAMP reactions additionally include a reverse transcriptase. Samples with potential template molecules are added to the reaction and incubated for 20 to 60 min at a constant temperature (e.g. 65° C.). The oligonucleotides act as primers for the reverse transcriptase, and additional oligonucleotides for the DNA polymerase are designed so the DNA products loop back at their ends. These, in turn, serve as self-priming templates for the DNA polymerase. In the presence of a few RNA template molecules, a chain reaction is set in motion, which then runs until the added reagents (in particular, the deoxynucleotide triphosphates) are used up.

LAMP assays or RT-LAMP assays may be a preferred embodiment due to their rapid nature, one-tube processing, and easy visualization of results without the need for expensive equipment or additional materials. In particular embodiments, the reagents for detection of the target comprise reagents for a colorimetric assay for detecting the target. Suitable colorimetric assays include, for example, colorimetric loop mediated isothermal amplification assays. Such embodiments allow for a facile visualization of whether or not the sample contains the target of interest. In some embodiments, the sample collection device contains reagents for a colorimetric loop mediated isothermal amplification (LAMP) assay. In embodiments wherein the nucleic acid is RNA, the sample collection device may contain reagents for a colorimetric RT-LAMP assay. In some embodiments, the reagents for a colorimetric LAMP assay (or colorimetric RT-LAMP assay) further include an indicator, which permits evaluation of a color change in the sample in the presence of sufficient nucleic acid (e.g. the target nucleic acid which the LAMP or RT-LAMP reagents are designed to detect). Suitable indicators include pH-sensitive indicators and metal-sensitive indicators. In preferred embodiments. pH-sensitive indicators (e.g. phenol red) may be used, due to their easy visualization with the naked eye.

In some embodiments, the reagents for detection of the target comprise reagents for a fluorescent assay for detecting the target. For example, the sample collection device may contain reagents for a fluorescent LAMP or fluorescent RT-LAMP assay. Any suitable fluorescent dye may be used in a fluorescent LAMP or fluorescent RT-LAMP assay to permit a fluorescent signal to be generated in the presence of sufficient nucleic acid.

In some embodiments, the reagents comprise oligonucleotides (e.g. primers) designed for detection of bacterial nucleic acid. In some embodiments, the reagents comprise oligonucleotides designed for detection of viral RNA. For example, the reagents may comprise oligonucleotides designed for detection of a viral upper respiratory infection selected from SARS-CoV2, SARS, a coronavirus, rhinovirus, influenza, respiratory syncytial virus, etc. In some embodiments, the reagents comprise oligonucleotides for detection of SARS-CoV-2 RNA.

Suitable reagents for detection of the target also include, for example, reagents for immunodetection of a target and/or a contaminant. In some embodiments, the reagents for detecting the target and/or the contaminant comprise reagents for immunoassays, which may use antibodies and/or antibody fragments to detect or measure a target or target analyte. In some embodiments, the immunoassay is an enzyme immunoassay, an ELISA (enzyme-linked immunosorbent assay, including direct ELISAs, indirect ELISAs, sandwich ELISAs and competitive ELISAs), an IEMA (immunoenzymometric assay), a radioimmunoassay, a fluoroimmunoassay, a chemiluminescent immunoassay (CLIA) and counting immunoassay (CIA).

In some embodiments, the sample is a biological sample obtained from a subject suspected of having an infection. In some embodiments, the subject is suspected of having SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, staphylococcus, aspergillus, or Streptococcus pyogenes. In some embodiments, the subject may be suspected of having a bacterial infection or a viral infection. For example, the subject may be suspected of having an upper respiratory infection. For example, the subject may be suspected of having a viral upper respiratory infection, including infection with SARS-CoV-2, a coronavirus, rhinovirus, influenza, respiratory syncytial virus, and the like.

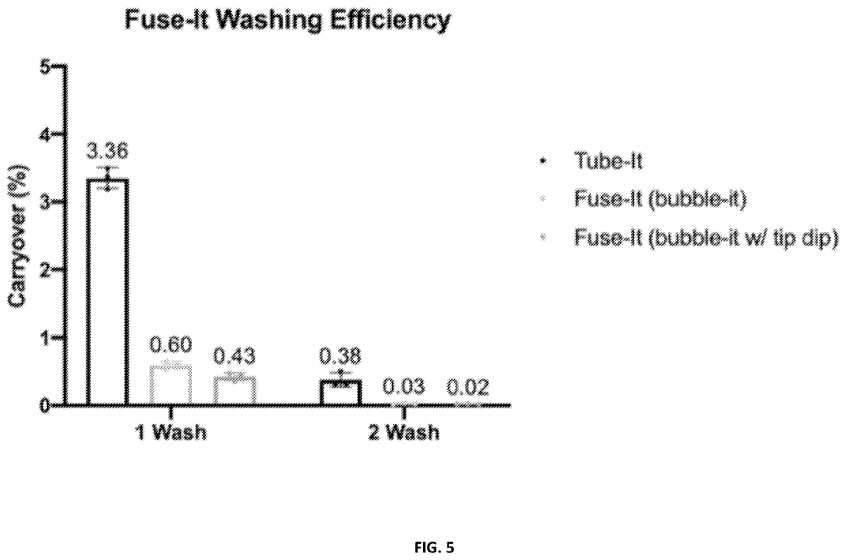

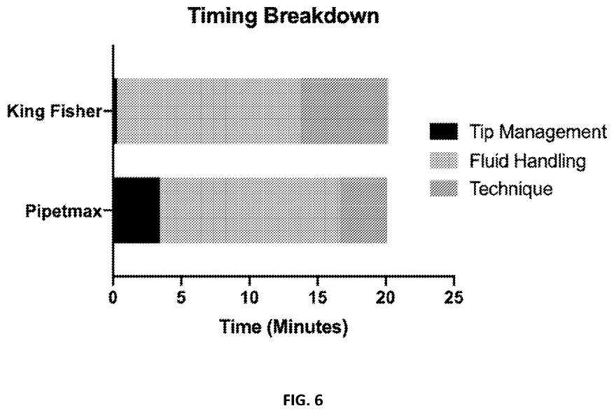

In some embodiments, the methods described herein are automated. For example, the method may be executed by a computer, wherein the computer comprises a processor and a memory. The memory may contain software which instructs the processor to execute a given task. Instruments may be used that contain one or more robotic components that permit movement of system components to facilitate automation of some or all of the steps of the method. In some embodiments, the systems and methods of the invention (e.g. FuseIt) and approaches to enhancing performance (e.g., Bubble-It, Tip-It) can all be accomplished using automation as demonstrated in preliminary data (see e.g. FIGS. 6, 7 and 8) using methods of the invention with, for example, a Gilson PIPETMAX liquid handling robot and associated Trilution and Protocol Builder softwares.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

### EXAMPLES

### Example 1

Performance of various embodiments of the methods described herein were assessed using absorbance measurements of tartrazine (yellow food coloring) concentrations. In this Example, the “sample” was tartrazine added to water with 0.05% Triton X100. The destination fluid (e.g. wash buffer) was water. Concentration was measured relative to the initial sample concentration using a DeNovix ds-11 spectrophotometer set to measure absorbance at 430 nm (i.e., near peak absorbance for tartrazine). The legend nomenclature is as follows. “Tube-It” refers to mimicking washing of beads in a tube where beads are held on the side of the tube while fluid is added and removed. Instead, using a 96-well plate here, this process is mimicked with beads held in the bottom of a 96-well plate with a magnet while fluid is added and removed. “Fuse-It” refers to a method of extracting or isolating at least one target from a fluid comprising a sample having multiple components, where the method comprises placing a container housing the fluid comprising the sample into a destination fluid that is sufficiently miscible with the fluid comprising the sample, wherein the container comprises an opening that permits movement of particles from the sample to the destination fluid, and the target is extracted or isolated by selectively moving particles from the fluid comprising the sample into the destination fluid. In some embodiments, the target is extracted or isolated by selectively moving the target from the fluid comprising the sample into the destination fluid. In some embodiments, the target is extracted or isolated by selectively moving contaminants (e.g. contaminant particles) from the fluid comprising the sample into the destination fluid. In this Example, refers to a minimal approach where a tip is a pipette tip and the tip is brought into the destination well and removed according to methods of the invention. Pre-aspiration and post-aspiration refer to pre- and/or post-aspiration of wash buffer (e.g. water). “Bubble-It” refers to pre-aspiration of air into the pipette tip, another method of the invention described herein. The phrase “Tip-Dip” refers to dipping the pipette tip briefly into a sacrificial well of wash fluid prior to entering the final destination fluid as a means to carryover surface contamination, which is another variation of methods of the invention.

FIG. 4 compares performance of Tube-It with variations of Fuse-It. Data demonstrated that Fuse-It with both pre- and post-aspiration can provide the same wash benefit as a single Tube-It wash, regardless of whether the beads are pre-concentrated to the end of the tip prior to entry into the wash fluid. Data also demonstrated the sizable benefit provided when Fuse-It is enhanced by Bubble-It. With Bubble-It, washing is >4 times better than Tube-It. After two washes, Fuse-It with Bubble-It is ˜16 times better (i.e., 4×4) than 2 Tube-It washes.

FIG. 5 illustrates, in a separate experiment, the benefit of removing surface carryover when performing Fuse-It and again confirms the relative benefit of Fuse-It compared to Tube-It. ˜30% of the contamination from Fuse-It with Bubble-It is from surface carryover on the outside of the tip. We envision replacing the tip-dip procedure with an oil overlay in the sample well will be advantageous for some future applications as well.

Together, these data demonstrate the functionality and benefits of all methods of the invention and multiple approaches to enhancing performance, with and without Bubble-It, and with and without Tip-Dip.

### Example 2

This Example compares the timing breakdown of running the same protocol for extracting nucleic acids with KingFisher technology and Fuse-It run using a Pipetmax.

A Macherey Nagel Nucleomag Tissue kit was used to process 16 samples of various concentrations of HeLa cells. The KingFisher utilized a magnet in a sheath technology for extraction and the Pipetmax utilized Fuse-It.

Each step of the protocol was timed and categorized into three portions. “Tip Management” refers to loading and ejecting tips. “Fluid Handling” refers to well mixing. “Technique” refers to bead collection and transfer.

As shown in FIG. 6, the Fuse-It technique proved to be twice as fast as the KingFisher magnet in a sheath technology. Importantly, an additional benefit of Fuse-It offers over other technologies like KingFisher is the ability to prep plates for running an extraction or PCR.

### Example 3

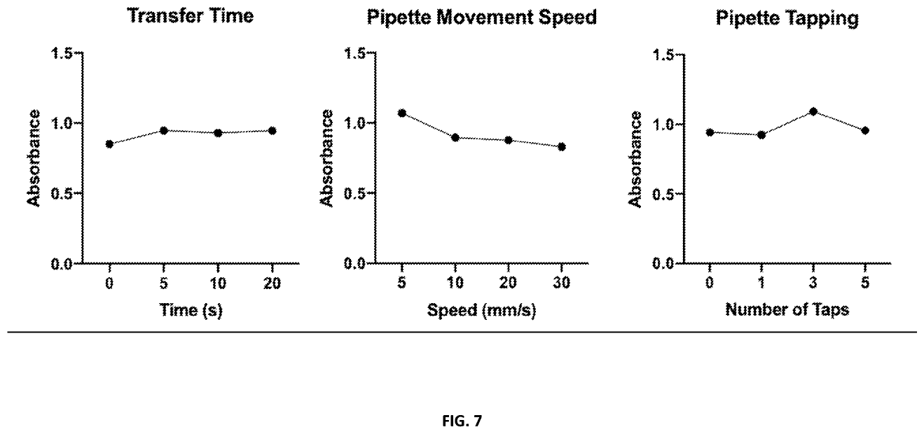

Performance of various embodiments of the methods described herein were assessed using several variables that improve bead transfer from a pipette tip with Fuse-It. These improvements were assessed using absorbance measurements of tartrazine (yellow food coloring) as shown in FIG. 7.

The sample was tartrazine added to PBS with 0.05% Triton with Nucleomag paramagnetic particles. FIG. 7 shows several variables to improve Fuse-It bead transfer from the pipette tip and demonstrates their impact on carryover. A higher absorbance is higher carryover.

Transfer time refers to how long the pipette stays in the well while PMPs transfer out of the tip. A slight increase in carryover was observed with a 5 second increase in transfer time. This is due to an increase in bead transfer within 5 seconds of time. There is no significant difference between 5 and 20 seconds.

Pipette movement speed refers to the speed at which the pipette enters and exits the well. A slower pipette entry movement speed results in higher carryover compared to a faster movement speed.

Pipette tapping refers to the action of moving a pipette tip that's situated at the bottom of the well up and down 1 mm. Pipette tapping can be utilized for PMPs with low magnetic responsiveness. There is no significant increase in carryover with an increasing number of taps.

### Example 4

This Example provides a purity comparison between the KingFisher and a “TSP” platform using the Macherey Nagel Nucleomag Tissue kit (i.e. the “Extraction Kit). TSP refers to “tip-based sample preparation,” which encompasses methods of the invention including Tip-It, Fuse-It and Bubble-It (using pipette tips).

The sample was tartrazine reconstituted in the provided kit buffers. TSP data was normalized to KingFisher which was set to 1. Individual step carryover refers to the carryover from the well the beads are collected from to the well that they are transferred into. Sample carryover refers to how much of the original sample is carried into the elution well. In this TSP protocol, a combination of Fuse-It and Bubble-It was used as the transfer mechanism. 24 μL and 10 uL refers to the volume of PMPs that were used with TMP and the Extraction Kit, the KingFisher ran 24 μL of PMPs (i.e. 24 μL and 10 μL refers to the volume of bead solution added to the sample).

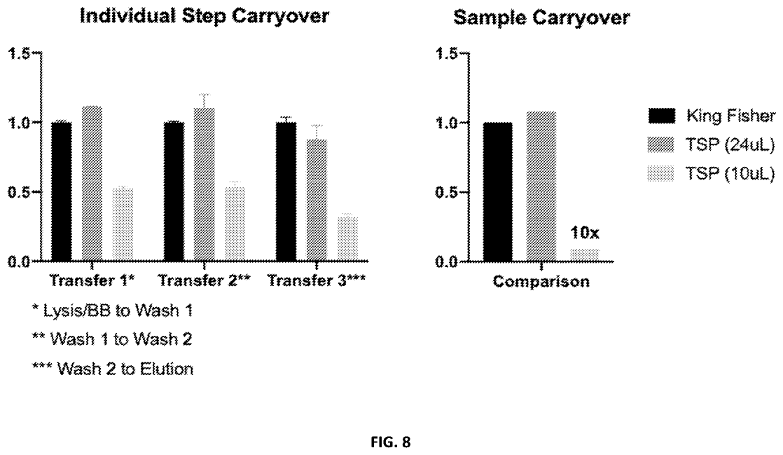

As shown in FIG. 8, the 24 uL TSP method is roughly equivalent to the KingFisher in carryover. The 10 uL TSP method of the invention is superior and results in ten times less carryover.

What is claimed is:

1. A method of extracting or isolating at least one target or component from a fluid comprising a sample having multiple components, the method comprising placing a container housing the fluid comprising the sample into a destination fluid that is sufficiently miscible with the fluid comprising the sample, wherein the container comprises an opening that permits movement of particles from the sample to the destination fluid.

2. The method of claim 1, wherein the target or component is extracted or isolated by selectively moving particles from the fluid comprising the sample into the destination fluid.

3. The method of claim 1 or claim 2, wherein the opening permits movement of at least one target or component from the sample to the destination fluid.

4. The method of claim 1 or claim 2, wherein the opening permits movement of at least one contaminant from the sample to the destination fluid.

5. The method of any one of the preceding claims, wherein the movement of the particles from the sample to the destination fluid occurs through diffusion, gravity, or acceleration.

6. The method of any one of the preceding claims, further comprising aspirating fluid into the container after the container is placed into the destination fluid.

7. The method of claim 6, wherein fluid is aspirated into the container before, during, and/or after movement of the particles from the container to the destination fluid.

8. The method of any one of the preceding claims, wherein the at least one target or component, if present in the sample, is bound to at least one paramagnetic particle (PMP) to form one or more target-PMP complexes or component-PMP complexes.

9. The method of claim 8, wherein the opening permits movement of the one or more target-PMP complexes and/or the one more component-PMP complexes from the sample to the destination fluid.

10. The method of claim 8 or claim 9, wherein movement of the one or more target-PMP complexes and/or the one more component-PMP complexes from the sample to the destination fluid occurs through application of a magnetic force to draw the one or more target-PMP complexes from the sample into the destination fluid.

11. The method of claim 4, wherein the at least one contaminant, if present in the sample, is bound to one or more paramagnetic particles (PMPs), to form one or more contaminant-PMP complexes.

12. The method of claim 11, wherein the opening permits movement of the one or more contaminant-PMP complexes from the sample into the destination fluid.

13. The method of claim 11 or 12, wherein movement of the one or more contaminant-PMP complexes from the sample to the destination fluid occurs through application of a magnetic force to draw the one or more contaminant-PMP complexes from the sample into the destination fluid.

14. The method of claim 10 or claim 13, further comprising aspirating fluid into the container before, during, and/or after application of the magnetic force.

15. The method of any one of the preceding claims, further comprising generating a pocket of air, oil, or gas proximal to the opening of the container prior to inserting the container into the destination fluid.

16. The method of any one of claims 1-15, wherein the at least one target or component is a cell.

17. The method of any one of claims 1-15, wherein the at least one target or component is an analyte.

18. The method of any one of the preceding claims, wherein the sample comprises a biological fluid.

19. The method of any one of the preceding claims, wherein the container is a pipette tip, a straw, or a capillary tube.

20. The method of any one of the preceding claims, wherein the destination fluid comprises a wash buffer, a precipitating buffer, a lysis buffer, an extraction buffer, an elution buffer, or a reaction mixture comprising reagents for detecting the target.

21. The method of any one of the preceding claims, further comprising placing the container into a cleansing fluid to reduce potential contaminants on an exterior surface of the container prior to placing the container into the destination fluid.

22. A system for isolating or extracting at least one target or component from a sample, the system comprising:
a. a container housing the sample, wherein the container comprises an opening;
b. a collection device comprising a destination fluid; and,
c. optionally, a sample comprising the at least one target or component, wherein the sample is sufficiently miscible with the destination fluid.

23. The system of claim 22, wherein the sample is a fluid and comprises the at least one target or component bound to one or more paramagnetic particles, thus forming one or more target-PMP complexes and/or one or more component-PMP complexes.

24. The system of claim 22, wherein the sample is a fluid and comprises at least one contaminant bound to one or more paramagnetic particles, thus forming one or more contaminant-PMP complexes.

25. The system of any one of claims 22-24, further comprising a magnet.

26. The system of any one of claims 22-25, further comprises a cleansing fluid.

27. The system of claim 26, wherein the cleansing fluid is housed within the collection device, wherein the cleansing fluid resides on top of the destination fluid such that the container housing the sample passes through the cleansing fluid prior to contacting the destination fluid.

28. The system of claim 27, wherein the cleansing fluid and the destination fluid are immiscible.

29. The system of any one of claims 22-28, wherein the at least one target or component or contaminant is a cell.

30. The system of any one of claims 22-28, wherein the at least one target or component is an analyte.

31. The system of any one of claims 22-30, wherein the sample comprises a biological fluid.

32. The system of any one of claims 22-31, wherein the container is a pipette tip, a straw, or a capillary tube.

33. The system of any one of claims 22-32, wherein the destination fluid is a wash buffer, an elution buffer, or a reaction mixture comprising reagents for detecting the target.

34. The method of any one of claims 1-21, further comprising a step of transferring the at least one target, the at least one component, the at least one contaminant, the one or more target-PMP complexes, the one more component-PMP complexes and/or the one more contaminant-PMP complexes, to another or different device or reagent mixture or reagent for further processing and/or analysis, identification, quantification or use.
